(19) United States
(12) Patent Application Publication
(10) Pub. No.: US 2024/0285367 A1
(43) Pub. Date: Aug. 29, 2024

(54) IMPLANTABLE MARKER

(71) Applicant: Thomas HESKE, Grafrath (DE)

(72) Inventor: Thomas HESKE, Grafrath (DE)

(21) Appl. No.: 18/573,501

(22) PCT Filed: Jun. 30, 2022
(86) PCT No.: PCT/EP2022/068034

§ 371 Date: Dec. 22, 2023

(30) Foreign Application Priority Data
Jun. 30, 2021 (DE) 10 2021 116 873.2

(51) Int. Cl. A61B 90/00

(52) U.S. Cl. CPC A61B 90/39; A61B 2090/3925

(57) ABSTRACT

The invention is an implantable marker for marking an intracorporeal tissue region of an animal or human, comprising at least one strand of a biocompatible material configured into a three-dimensional shape impressed upon it during a shaping process. The three-dimensional shape is formed by external mechanical force, applied to the at least one strand once the mechanical force ceases, the strand comprises at least two fixed strand eyes. Each of which is formed by at least one winding of the strand and has a fixed relative spatial position with respect to at least one other eye once the mechanical force ceases. A winding plane is associated with the strand eyes having winding planes relative to each other to form an angle α which is not equal to 0°.

### CROSS-REFERENCE TO RELATED APPLICATIONS

Reference is made to PCT/EP2022/068034, filed on Jun. 30, 2022 and German Patent Application No. 10 2021 116 873.2 filed Jun. 30, 2021, which is incorporated herein by reference in its entirety.

### BACKGROUND OF THE INVENTION

### Field of the Invention

The invention relates to an implantable marker for marking an intracorporeal tissue region of an animal or human, which comprises at least one strand, of a biocompatible material and has a three-dimensional shape impressed by a shaping process, which three-dimensional shape is adopted by the at least one strand, after a three-dimensional a force is applied to the strand by external mechanical force, and when the mechanical force ceases, the three-dimensional shape impressed upon the strand comprises at least two fixed strand eyes, with a first and a second strand eye, each of which is formed by at least one winding of the strand of biocompatible material and of which the relative spatial position of the at least two strand eyes with respect to one another are different once the mechanical force ceases.

### Description of the Prior Art

Generic implantable markers are used to identify tumors in soft tissue of a human or animal. For example, after a breast biopsy, a marker is often inserted into the site of tissue removal using a cannula and ejected from the distal side of the cannula using a stylet once the desired location has been reached. The intracorporeally placed marker remains stationary and offers a doctor the opportunity to locate an area of tissue at least one of treated and diagnosed with the aid of an imaging procedure, preferably using ultrasound images, and to observe it over the long term.

A tissue marker for human tissue is disclosed by U.S. Pat. No. 6,053,925. It has two twisted wires made of shape-memory metal with distal end regions which assume a ring or coil shape as an anchoring structure within the tissue. After intracorporeal localization of the marker, the twisted wires protrude proximally from the body area and thus define a direct trajectory to the marked tissue site.

The published patent application US 2005/0059888 A1 describes a marker marking the location of a biological absorber body placed intracorporeally. The marker is a material detectable by mammography, radiology and ultrasound examination, for example a wire attached to the absorber body.

The published patent application US 2001/0023322 A1 contains a cannula-like positioning unit for an intracorporeally insertable marker. The marker has a shape-memory metal wire which, after intracorporeal positioning, deforms into a ring or coil manner at least at the tip of the wire for the purpose of providing a fixed positioning in a tissue area to be marked.

Lastly, EP 1 871 266 B1 contains a generic marker for human or animal tissue, which is made of a pre-programmable material, preferably a nickel-titanium alloy, in the form of a ring, which returns to the pre-programmed ring shape once a longitudinal extension forced upon it ceases.

All known, generic markers, in particular the ring markers described above, are capable of reflecting ultrasonic waves due to their material properties and thus represent ultrasonic reflectors. But the known markers are visually perceptible to varying degrees on the ultrasound images that can be generated, depending on the direction of sonication along which the ultrasonic waves interact with the marker. For example, ultrasonic waves of an ultrasonic wave field propagating mostly 2-dimensionally in a fan shape hit a ring marker having a ring plane oriented orthogonally to the fan plane of the propagating ultrasonic waves which causes a maximum of two point-like ultrasonic reflection image signals to appear, which correspond to the intersection points of the fan plane with the ring marker. When the fan-shaped ultrasonic wave field is dynamically swept over the ring marker in the constellation described above, ideally a point-like ultrasonic reflection image signal first appears, which then splits into two separate ultrasonic reflection image signals causing the mutual distances to first increase and then to decrease again until only a point-like ultrasonic reflection image signal can be seen again. If, on the other hand, the fan-shaped ultrasonic wave field is ideally oriented parallel to the ring plane and interacts with the ring marker, a complete ring shape appears on the ultrasound image and is clearly visible to the diagnostician.

During an ultrasonic wave examination, intense ultrasonic wave reflections frequently occur, particularly in areas with proportions of dense tissue materials, and are visible in the ultrasound image as significantly perceptible reflection events and visually compete with the ultrasound reflection image signals originating from the implanted markers. Only in the aforementioned ideal case of sonication of a ring-marked tissue area parallel to the ring plane does the ring shape, which can be clearly distinguished from natural tissue structures due to its Euclidean three-dimensional shape, enable clear visual identification of the location and position of the marker.

A great deal of experience is therefore needed to clearly recognize such markers, which are known per se and the dimensions of which are in the range of a few millimetres, physically and in a manner spatially resolved within the biological tissue environment.

DE 10 2019 210 963 A1 describes an implantable marker that provides, along a strand made of biocompatible material, at least two fixed strand eyes, which are each integrally connected to one another via a strand portion.

### SUMMARY OF THE INVENTION

The invention is an implantable marker for marking an intracorporeal tissue region of an animal or human, which marker comprises at least one strand, which is made of biocompatible material and has a three-dimensional shape impressed upon it during a shaping process, causing the three-dimensional shape to be adopted by the strand, after a three-dimensional shape is applied to the strand by an external mechanical force. Once the mechanical force ceases, the development is that the visibility and identification of the implanted marker for a physician during an ultrasound examination are considerably improved.

According to the invention, an implantable marker for marking an intracorporeal tissue region of an animal or human, comprises at least one strand, which is made of biocompatible material and has a three-dimensional shape impressed upon it during a shaping process. The three-dimensional shape is adopted by the strand after a three-dimensional shape is to the strand by external mechanical force. Once the mechanical force ceases, the three-dimensional shape applied to the strand comprises at least two fixed strand eyes which are a first and a second strand eye. Each eye, which is formed by at least one winding of the strand and each of which can be associated with a winding plane, causes the at least two strand eyes to be formed in a manner comparable with shaped eyes predetermined by the shape of a figure of eight. The eyes are connected to one another via a common point of contact at which the winding planes of the at least two strand eyes are oriented inclined to one another by the angle α, and the winding planes of the at least two strand eyes form an angle α unequal to 0°.

The term “fixed strand eye” is borrowed from knotology and describes a simple geometric shape characterized by the formation of a loop along the strand. For this purpose, the strand is helically wound at least once and has a helical pitch in which the strand preferably touches itself in the overlapping area of the winding. In contrast to a closed ring shape, a fixed eye does not appear as a straight line with a constant line thickness when exposed to ultrasonic waves from the side, but appears wedge-shaped or double-wedge-shaped with the maximum wedge width corresponding to twice the width of the strand in the case of a single helical winding. Accordingly, an ultrasonic signal is formed more clearly when a fixed strand eye composed of several helical windings is exposed to sonic waves from the side.

Alternatively, the term “fixed strand eye” is to be understood to be a loop formed in a winding plane, with a strand end lying in the winding plane of the loop.

The term “winding plane” is to be understood to be the spatial plane in the sense of the invention that can be associated with a fixed eye and contains the area that, in perpendicular projection onto the fixed eye, is spanned by the latter.

The implantable marker according to the invention has at least two fixed strand eyes which are each formed along the strand by a helical winding of the strand. Not necessarily, but advantageously, the at least two fixed strand eyes have the same shape and dimensions. Of course, eye shapes that deviate from the circular shape, such as oval or elliptical fixed eyes, are also conceivable. The shapes and dimensions of the strand eyes arranged along the strand can also differ from one another. In addition, the spatial position and association of the fixed strand eyes and also the orientation of their winding direction can be selected uniformly or individually.

The marker designed according to the invention is based on improving of its detectability and identifiability by use of common ultrasonic wave diagnostic technology in such a way that the at least two fixed eyes are inclined or aligned relative to each other in such a way that, in the case of orthogonal exposure of the marker to sonic waves relative to the winding plane of one fixed strand eye, the other fixed strand eye is preferably exposed in alternation to the ultrasonic waves along its winding plane, in order to appear at least partially and preferably as a complete ring or loop contour on the ultrasonic image by use of ultrasonic wave detection.

Of particular importance for improving detectability of the marker by use of ultrasonic waves is a three-dimensional shape that is as compact as possible, that is the ultrasonic wave reflection signals originating from the marker, which are visualized on a monitor for a diagnostician, should originate from a small spatial area, that is elongate marker shapes, in which reflection structures are arranged next to each other in serial sequence, which proves to be rather difficult to detect. The preferably direct connection of two fixed eyes, which are integrally connected to each other via a contact point, comparable to the intersection point at which the shaped eyes of a figure eight meet, as well as the preferably orthogonal orientation of both winding planes of the strand eyes integrally connected to each other at the contact point, leads to a dynamically changing reflection image or pattern on an observation monitor during ultrasonic wave detection with a fan-shaped ultrasonic field, which is panned over the intracorporeally placed marker The reflection image or pattern is characterized, depending on the spatial orientation of the marker relative to the ultrasonic wave field, by a dynamically changing ring structure in which the technical shape is interpreted as a clear indication of the position of the marker, in contrast to reflection signal images that are based on intracorporeal biological tissue structures. Due to the compact spatial arrangement of the at least two strand eyes, the detectability of the marker is particularly obvious to the person performing the ultrasound examination due to the dynamic visibility resulting from the ultrasonic field panning.

The at least two strand eyes are arranged as compactly as possible along the strand if the strand has one strand length and two strand ends, in such a way that one of the two strand ends is arranged in the winding plane of the first strand eye and forms the first strand eye together with one half of the strand length and the other of the two strand ends is arranged in the winding plane of the second strand eye and forms the second strand eye together with the other half of the strand length. Preferably in this case, both strand ends are each arranged laterally adjacent to and opposite the strand in the area of the middle of the strand length. In a particularly preferred embodiment, the winding planes of both strand eyes are oriented orthogonally to each other. This constellation can be achieved by orienting both strand eyes at 90° to each other from a common plane at the point of contact. In this constellation, both strand eyes are arranged without overlap. Of course, suitable angles deviating from 90°, between 0° and 180°, are also possible, excluding the critical angles, although the orthogonality of the at least two angular planes to each other or deviating from this by an angle of ±30° represent particularly suitable three-dimensional shapes for the forming of the implantable marker according to the invention.

In particular, good detection properties can be recorded for the marker if both strand eyes have an angle α with 60°≤α≤120°. In the case of 60°≤α≤90°, both strand eyes overlap in relation to the orthogonal projection onto their respective winding planes.

In a further embodiment, the at least two strand eyes are designed and arranged in such a way that, in orthogonal projection onto one of the two strand eyes, the other strand eye is arranged in the middle of the first strand eye. This constellation is achievable if, based on the constellation explained above with α=90°, both strand eyes are additionally rotated by 90° at their point of contact in the longitudinal axis of the strand, that is the strand is rotated torsionally by 90°.

An alternative embodiment of the implantable marker according to the invention comprises at least two strand eyes which are connected to each other via a curved strand portion which an arc plane being associated which forms an angle β with each of the winding planes of the at least two strand eyes, which angle is not equal to 0°, and the winding planes of the at least two strand eyes form an angle α which is not equal to 0°. The curved strand portion is preferably semi-circular. Preferably, the at least two strand eyes have the same shape and the same dimensions. In this case, the curved strand portion is shaped and dimensioned identically to one half of one of the strand eyes.

In principle, an angle of 50°≤β≤70° is suitable for B. If the curved strand portion forms an angle β=60° with the winding planes of both strand eyes and if, as mentioned above, the curved strand portion is shaped and dimensioned equally to half of the two strand eyes, both strand eyes together with the curved strand portion define the shape of an equilateral triangle. If a marker designed in this way is detected by dynamically panning a fan-shaped ultrasonic wave field, the reflection signals that can be visually perceived on a viewing device emerge as a dynamically appearing three-dimensional ring structure with an unmistakable conciseness.

The marker, which preferably is biocompatible, metallic shape-memory material over the entire length of its strand, is inserted into a hollow cannula for the purpose of implantation by exerting a mechanical force which transforms the marker from its three-dimensional shape impressed into the shape-memory material into a largely rectilinear strand shape. The cannula intended for the implantation procedure typically has a cannula diameter that is slightly larger than the strand diameter so that the substantially rectilinear strand can be removed from the hollow cannula along the length of the hollow cannula at the distal end thereof using a stylet. When the strand emerges from the hollow cannula, it deforms into its pre-impressed three-dimensional shape, forming at least two fixed strand eyes that are integrally connected to each other.

In order to minimize the risk of lesions for the tissue to be marked and at the same time to optimize the detection and identification properties of the marker by use of ultrasound detection, the marker has the most compact possible three-dimensional shape, that is the two strand ends which delimit the strand on both sides are each arranged in a winding plane that can be associated with the strand eyes and, in the case of two fixed strand eyes, form a fixed strand eye together with half of the strand length in each case.

In the case of the formation of three or more strand eyes along the strand, the strand ends are arranged within the winding planes of the fixed strand eyes formed at each end of the strand. In particularly preferred embodiments for designing the implantable marker according to the invention, the at least two fixed strand eyes along the strand are directly connected to one another or merge directly into one another, that is in a force-free state the implantable marker has exclusively curved strand portions forming fixed strand eyes. Alternatively, two strand eyes are integrally connected to each other via a curved strand portion, wherein the curved strand portion has approximately the same dimensions and half of the shape of both strand eyes.

In order to increase the ultrasonic reflectivity of the strand made of biocompatible material, the strand surface is modified or treated so that it has an ultrasonic wave-reflecting surface structured, for example, by roughening the material, at least in some areas.

Alternatively or in combination with the above measures for increasing the ultrasonic wave reflectivity, it is possible to manufacture the strand from a center wire and at least one additional single wire which is helically wound around the center wire, whereby the outwardly appearing strand surface is provided through a helical winding structure of at least one single wire. At least the center wire or the at least one individual wire is made here of a metallic shape-memory material, preferably NiTi (Nitinol), NiTiCu, CuZn, CuZnAl or CuAlNi.

Preferably, the strand has a cross-section of 0.1 to 0.8 mm. In the case of winding at least one single wire around a center wire, wire cross-sections in the range from 0.1 mm to 0.5 mm are suitable.

The preferably ring-shaped strand eyes typically have a ring inner diameter of 2 to 3 mm and a ring outer diameter of 3 to 5 mm.

Cannulas with cannula sizes between 14 G and 18 G are particularly suitable for implanting the marker according to the invention.

The implantable marker according to the invention is explained in greater detail below with reference to the following figures on the basis of various exemplary embodiments.

### BRIEF DESCRIPTION OF THE DRAWINGS

The invention is described below by way of example on the basis of exemplary embodiments with reference to the drawing, without limiting the general idea of the invention.

The drawing shows:

FIG. 1 is a schematic diagram illustrating the ultrasonic detectability of an ultrasonic marker formed in accordance with the invention;

FIGS. 2a, b are a) is a side view of a first exemplary embodiment; and
b) is an oblique view of the first exemplary embodiment

FIGS. 3a, b are a) is a plan view of a second exemplary embodiment;
b) is an oblique view of the second exemplary embodiment;

FIGS. 4a, b are two side views of a third embodiment rotated by 90°;

FIG. 5 is a perspective view of a fourth exemplary embodiment;

FIGS. 6 a, b are side view illustrations of a fifth exemplary embodiment; and

FIGS. 7 a, b are side view illustrations of a sixth exemplary embodiment.

### DETAILED DESCRIPTION OF THE INVENTION

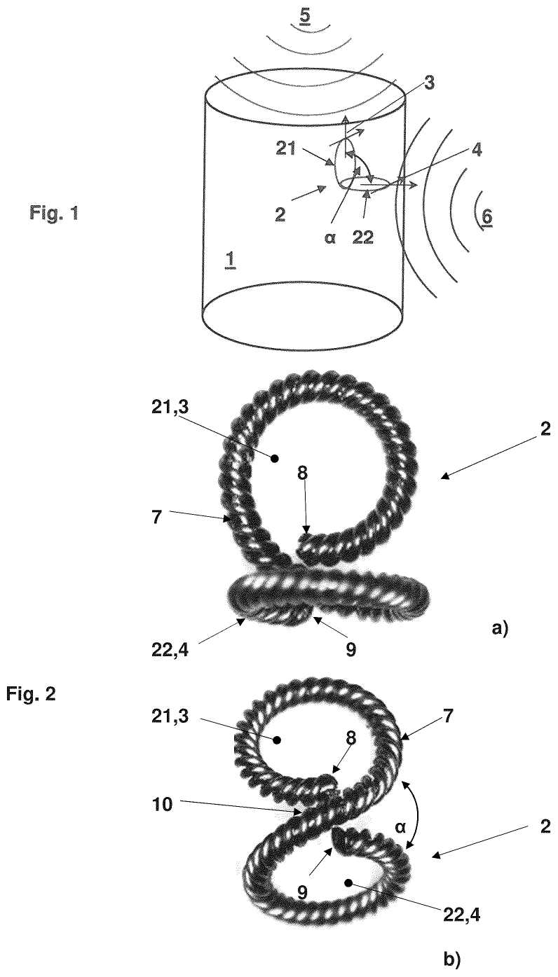

FIG. 1 shows a schematic representation of a marker 2 implanted within a tissue area 1. The marker assumes its impressed three-dimensional shape once external mechanical forces cease and has at least two strand eyes 21, 22, each of which is associated with a winding plane 3, 4, which together enclose an angle α≠0, preferably 90°.

In the case of ultrasonic wave detection, a ring-shaped reflection pattern is displayed on an ultrasonic image of the marker 2 shown schematically in FIG. 1, in each case in interaction with ultrasonic waves 5, 6, the propagation direction of which is oriented orthogonally to one of the two winding planes 3, 4. In the case of ultrasonic waves 5, the propagation direction of oriented orthogonally to the winding plane 4 and therefore passes through the strand eye 21 in the longitudinal direction causing the strand eye 21 to be displayed in a ring shape on a corresponding ultrasonic image. In the case of ultrasonic waves 6, on the other hand, the strand eye 22 is shown in a ring shape on a corresponding ultrasound image. In both cases, a Euclidean geometric shape, that does not occur in the natural tissue area, appears on an ultrasound image display and draws the attention of the person carrying out the examination to the existence of the intracorporeally located marker 2.

In the implanted state, that is in a state in which the marker 2 assumes its three-dimensional shape inherently impressed in the material, the marker 2 made of a single strand-like material forms a largely self-contained three-dimensional shape, that is the strand ends of the strand forming the marker 2 are preferably each located along a winding plane 3, 4. Specific three-dimensional shapes according to the invention for forming the implantable marker 2 can be seen in the further figures.

FIGS. 2 a, b show a marker 2 from two different views. The marker is made from a strand 7 and includes two strand eyes 21, 22. The two strand ends 8, 9 of the strand 7 are located in the region of the strand middle area 10, in each case opposite the strand 7, wherein the strand end 8 is arranged within the winding plane 3 which can be associated with the strand eye 21 and which corresponds to the drawing plane as shown in FIG. 2a, and the strand end 9 is arranged within the winding plane 4 which can be associated with the strand eye 22 and which is oriented orthogonally to the drawing plane according to FIG. 2a. The winding planes 3, 4 of the strand eyes 21, 22 form an angle α. See FIG. 2b, which is 90°.

The strand 7 of the marker 2 preferably has a center wire around which a single wire is helically wound. Of course, two or more wires can also be wound around the center wire.

FIGS. 3 a, b each show, from different directions, a marker 2 made from a strand 7 and formed in accordance with the invention, wherein the strand 7 forms two fixed strand eyes 21, 22. FIG. 3a shows that the winding plane 3 which can be associated with the first strand eye 21 which corresponds to the sheet or drawing plane. The winding plane 4 of the second strand eye 22, on the other hand, extends orthogonally to the winding plane 3, wherein the second strand eye 22 is arranged in the middle of the first strand eye 21 in a projection onto the latter.

In this case too, ultrasonic waves, that interact with the marker 2 parallel to the winding plane of one of the two strand eyes 21, 22, make it possible to display in the ultrasonic image the complete eye shape of the strand eye oriented in the direction of transmission. Preferably, the strand eyes 21, 22 are each ring-shaped or loop-shaped.

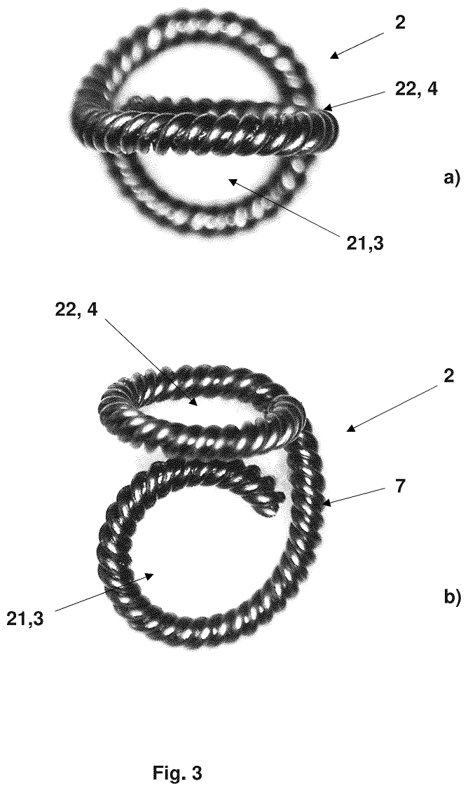

FIGS. 4 a, b each show a marker 2 in respective side views rotated through 90° relative to each other.

The strand 7 has a first fixed strand eye 21 with a winding plane 3 oriented orthogonally to the drawing plane. The strand end 8 shown in FIG. 4b is arranged within the winding plane 3.

The second strand eye 22 extends in a three-dimensional, slightly helical winding manner above the first strand eye 21, which forms the second strand eye 22 when seen in the side view illustrated in FIG. 4b. The winding plane 4, which can be associated with the second strand eye 22, is oriented orthogonally to the sheet or drawing plane in FIG. 4a or corresponds to the sheet or drawing plane in FIG. 4b.

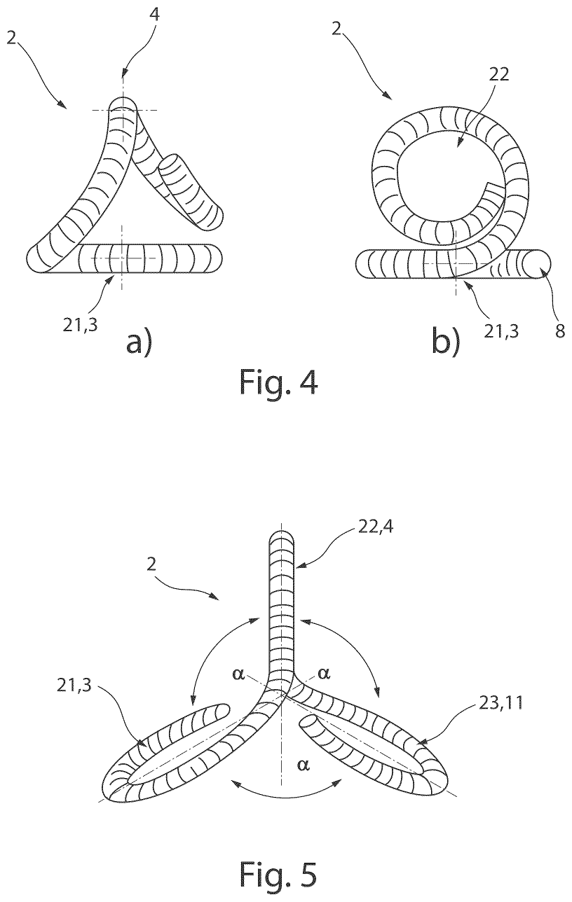

FIG. 5 illustrates an embodiment of a marker 2 formed in accordance with the invention, forming three strand eyes 21, 22, 23, the associated winding planes 3, 4, 11 of which in each case form an angle α of 120°. In this case, the strand end 8 is located within the first winding plane 3 and the strand end 9 is located within the winding plane 11.

In contrast to the exemplary embodiments illustrated above, the marker 2 illustrated in FIG. 5 enables a ring-shaped or loop-shaped image to be provided on an ultrasound image when sonic waves are propagated in each of the winding planes 3, 4, 11, which improves the spatial detectability and also the identifiability of the marker 2 compared to the markers described above, each with two strand eyes.

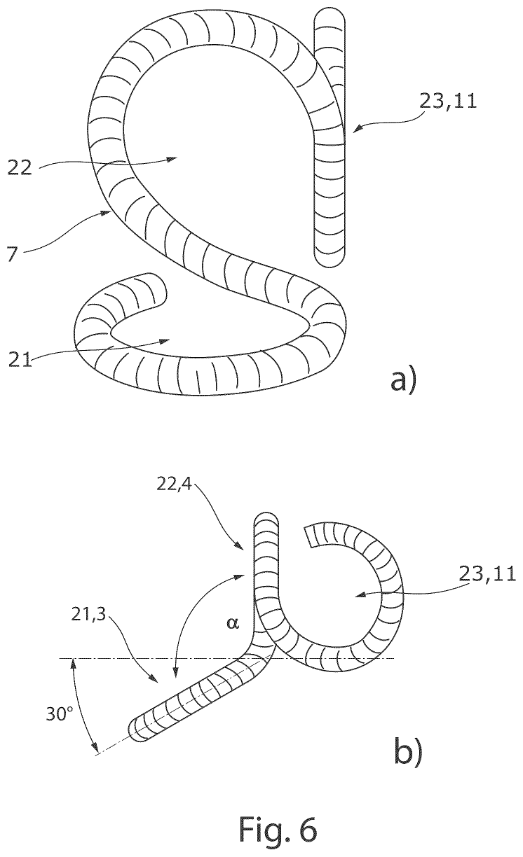

FIGS. 6 a, b show a further embodiment of a marker 2 from two different viewing directions. The strand 7 of the marker 2 includes three strand eyes 21, 22, 23, associated with one each of the winding planes 3, 4, 11. The winding planes 3, 4 associated with the first and second strand eyes 21, 22 form an angle α, which is 120° in the exemplary embodiment shown. Three-dimensional shapes deviating from this are also conceivable, for example with α=90°. The winding plane 11 associated with the third strand eye 23 is tilted or oriented orthogonally to the second winding plane 4.

In this case too, the marker 2, which is designed in accordance with the invention, generates ring-shaped or loop-shaped ultrasonic reflection images on an ultrasonic image, in each case with sonic wave propagation in the three winding planes 3, 4 and 11.

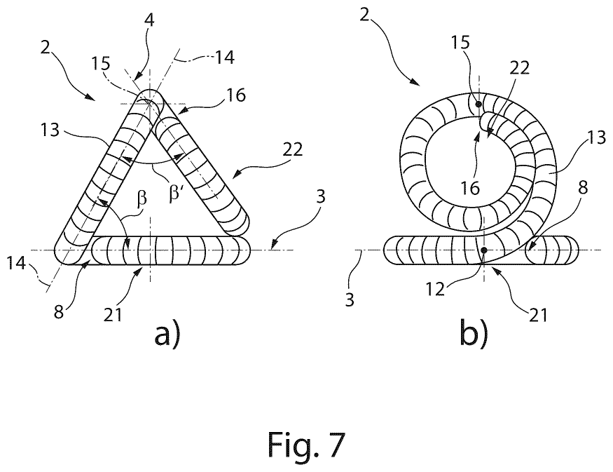

FIGS. 7a and b each respectively show a marker 2 in side views rotated through 90° relative to each other, comparable to the exemplary embodiment illustrated in FIG. 4 a and b.

The first fixed strand eye 21 has a winding plane 3 which is oriented orthogonally to the drawing plane. The strand end 8 of the first strand eye 21 shown in FIG. 7b is arranged within the winding plane 3 and is directly adjacent to a bending point 12, at which the first strand eye 21 merges integrally into a curved strand portion 13, which is semi-circular ring-shaped and spans an arc plane 14. The arc plane 14 forms an angle β with the winding plane 3 of the first strand eye 21, the angle preferably being 60°. The upper end of the curved strand portion 13 shown in the illustration is integrally connected at the bending point 15 to the second strand eye 22 with the associated winding plane 4 forming an angle β′ with the curved plane 14 The angle preferably corresponds to the angle β. The strand end 16 of the second strand eye 22 is located in the winding plane 4 and is directly adjacent to the second bending point 15.

The first and second strand eyes 21, 22 are preferably of the same shape and approximately the same size. In this case, the curved portion 13 corresponds to one half of the shape of one of the two strand eyes 21, 22. The marker 2 thus comprises a three-dimensional body which can be characterized by an equilateral triangle and which is capable of generating a very concise reflection pattern in the ultrasonic echo image. The dynamic visibility of the pattern appears three-dimensionally on a monitor when a fan-shaped ultrasonic field is panned over the marker.

The first and second strand eyes 21, 22 do not necessarily have to have the same dimensions. Rather, the curved portion 13 should be adapted appropriately in shape and size to the second strand eye 22 shown in FIG. 7 a, b in order to form a self-contained three-dimensional triangular structure.

### LIST OF REFERENCE SIGNS

1 tissue area
2 marker
3 winding plane
4 winding plane
5 ultrasonic waves
6 ultrasonic waves
7 strand
8 strand end
9 strand end
10 strand length middle
11 winding plane
12 bending point
13 curved portion
14 arc plane
15 bending point
16 strand end
21 strand eye
22 strand eye
23 strand eye
α angle
β, β′ angle

1-16. (canceled)

17. An implantable marker for marking an intracorporeal tissue region of an animal or human, comprising:
at least one strand of biocompatible material configured into a three-dimensional shape comprising at least first and second fixed strand eyes, each strand eye includes at least one winding of the biocompatible material within a winding plane of one of the first and second strand eyes; and
the at least first and second strand eyes are configured into a figure eight, are connected together via a common point of contact at which the winding planes of the at least first and second strand eyes meet and form an angle α which is not equal to 0°.

18. The implantable marker according to claim 17, wherein each strand of the biocompatible material has a strand length and two strand ends, one of the two strand ends is positioned in the winding plane of the first strand eye and contains one half of the length of at least one strand of biocompatible material, and another of the two strand ends is positioned in the winding plane of the second strand eye and contains another half of the strand length of the at least one strand of the biocompatible material.

19. The implantable marker according to claim 18, wherein each strand end of the first strand eye and the second strand eye is positioned adjacent to and opposite an area at a midpoint of the strand length.

20. The implantable marker according to claim 17, wherein at least the first and second strand eyes are positioned orthogonally to project onto one of the two strand eyes and another strand eye is arranged without overlapping the first strand eye.

21. The implantable marker according to claim 20, wherein α: 90°≤α≤120°.

22. The implantable marker according to claim 17, wherein the at least first and second strand eyes are positioned so that upon an orthogonal projection onto one of the first and second strand eyes, the other strand of the first and second strand eyes is positioned at a midpoint of the other strand eye.

23. The implantable marker for marking an intracorporeal tissue region of an animal or human, comprising:
at least one strand of biocompatible material configured into a three-dimensional shape comprising at least first and second fixed strand eyes, each strand eye includes at least one winding of the biocompatible material within a winding plane of one of the first and second strand eyes; and
the at least first and second strand eyes are configured into a figure eight, are connected together via a curved strand portion and the winding planes of the at least first and second strand eyes meet and form an angle α which is not equal to 0°.

24. The implantable marker according to claim 23, wherein the curved strand portion is semi-circular.

25. The implantable marker according to claim 24, wherein the winding planes of at least one of the two strand eyes forms an angle β of 50°≤β≤70° with the arc plane.

26. The implantable marker according to claim 25, wherein β=60°.

27. The implantable marker according to claim 25, wherein the winding plane of at least one of the two strand eyes forms an angle β in which 50°≤β≤70° with the arc plane.

28. The implantable marker according to claim 18, wherein:
the strand length of the at least one strand of biocompatible material equals a sum of a length of the curved strand portions of the two strand eyes;
one of the two strand ends of the at least two strand eyes are positioned in the winding plane of the first strand eye; and
the other of the two strand ends is positioned in the winding plane of the second strand eye.

29. The implantable marker according to claim 24, wherein the biocompatible material of the at least one strand reflects ultrasonic waves.

30. The implantable marker according to claim 17, wherein the at least one strand of biocompatible material has a surface in at least some areas of the at least one strand which reflects ultrasonic waves.

31. The implantable marker according to claim 17, wherein the at least one strand of biocompatible material includes a center wire and at least one single wire is wound helically around the center wire and comprises a metallic shape-memory material.

32. The implantable marker according to claim 17, wherein the strand made of biocompatible material comprises at least one wire made of a metallic shape-memory material selected from the following group: NiTi (“Nitinol”), NiTiCu, CuZn, CuZnAl or CuAlNi.

33. A method of deploying an implantable marker according to claim 17, wherein the at least one strand is three-dimensionally shaped by an external mechanical force, is without any strand eyes, is positioned in a hollow cannula and exerts the external mechanical force on the at least one strand, and immediately after removal of the at least one strand from the hollow cannula and removal of the external mechanical force, the at least two strand eyes are formed independently.

34. The implantable marker according to claim 33, wherein the hollow cannula is selected from sizes ranging from 14 G to 18 G.
